(19) United States
(12) Patent Application Publication
(10) Pub. No.: US 2004/0175314 A1
(43) Pub. Date: Sep. 9, 2004

(54) Self-reducing anthraquinone compounds

(76) Inventors: Mark Andrew Horsham, Mascot (AU); Andrew David Scully, North Ryde (AU); James Keith Gerard Murphy, Baulkham Hills (AU)

Correspondence Address: Robert L Price, McDermott Will & Emery, 600 13th Street NW, Washington, DC, 20005-3096, US

(21) Appl. No.: 10/472,549

(22) PCT Filed: Mar. 22, 2002
(86) PCT No.: PCT/AU02/00341

(30) Foreign Application Priority Data
Mar. 23, 2001 (AU) PR 3937

(51) Int. Cl. B01D 53/34; C07D 241/04; C07D 265/30

(52) U.S. Cl. 423/219; 544/156; 544/380

(57) ABSTRACT

A method of scavenging oxygen in an atmosphere or liquid comprising the steps of: (i) treating an anthraquinone compound according to the formula: (I) wherein X1-X4 and R1-R4 are as defined in the claims, with predetermined conditions so as to reduce the anthraquinone compound to a reduced form oxidizable by oxygen; and ii) exposing the atmosphere or liquid to said compound; such that at least a portion of teh oxygen in teh atmosphere or liquid is removed through oxidation of the reduced form of the anthraquinone compound, and wherein steps i) and ii) may be carried out in either order.

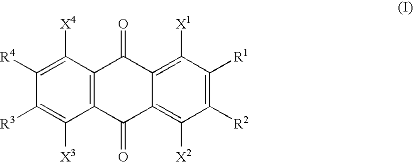

### FIELD OF THE INVENTION

This invention relates to oxygen scavenging compositions comprising a class of self-reducing anthraquinone compounds, for use in, for example, food and beverage packaging to scavenge unwanted oxygen, which either remains within the package following the packaging of the food or beverage or otherwise enters the package by permeating through the packaging material. The self-reducing anthraquinone compounds may also be incorporated into packaging materials, to prevent oxygen from permeating through the packaging material to enter the inside of a package. In addition, the self-reducing anthraquinone compounds may be incorporated into packaging materials to reveal leaks in packages or to indicate package damage caused by handling or tampering.

### BACKGROUND TO THE INVENTION

A wide variety of foods, beverages and other materials are susceptible to loss in quality if they are exposed to significant amounts of oxygen during storage. The damage can arise from, for example, chemical oxidation of the product and/or microbial growth. In the field of packaging, such damage has been traditionally addressed by generating relatively low-oxygen atmospheres by vacuum packing and/or inert gas flushing. However, these methods are not generally applicable for various reasons. For example, the fast filling speeds commonly used in the food and beverage industries often prevent effective evacuation of, or thorough inert gas flushing of, food and beverage packages, and neither evacuation or inert gas flushing provides any residual capacity for removal of oxygen which may have desorbed from the package contents or entered the package by leakage or permeation. As a consequence, there has been much interest in the identification and development of chemical techniques for generating low-oxygen atmospheres.

In Australian Patent No. 672661 (the entire disclosure of which is incorporated herein by reference), the present applicants describe novel oxygen scavenging compositions comprising a source of labile hydrogen or electrons and a reducible organic compound such as an anthraquinone (AQ), which may be readily activated or “triggered” (ie brought to their oxygen scavenging form) as required by exposure to, for example, ultraviolet (UV) light. The oxygen scavenging compositions, once activated, are capable of scavenging oxygen from an oxygenated atmosphere or liquid in substantial darkness for periods ranging from up to a few minutes or hours to over 100 days.

The oxygen scavenging compositions described in Australian Patent No. 672661, include a source of labile hydrogens or electrons to allow activation of the reducible organic compound by, for example, photoreduction. The source of labile hydrogen or electrons may be a compound such as a salt of a sulfonic or carboxylic acid or a compound having a hydrogen atom bonded to a carbon atom which is, in turn, bonded to a nitrogen, sulfur, phosphorus, or oxygen atom or, where the composition includes a polymer (eg typical polymers of food and beverage packaging such as ethylene vinyl acetate) which may or may not be covalently linked to the reducible organic compound, the source of labile hydrogen or electrons may be borne on the polymer. Australian Patent No. 672661 also teaches the possibility of providing the source of labile hydrogen or electrons on the reducible organic compound itself (eg a sodium sulfonate salt of the reducible organic compound). This kind of reducible organic compound might be regarded as being “self-reducing” upon exposure to, for example, UV light, and may be particularly suitable when the composition includes a non- or poorly-hydrogen/electron donating polymer such as polyethylene terephthalate (PET).

The present applicants have now identified certain classes of anthraquinone compounds, bearing hydrogen or electron donor substituents, which self-reduce when subjected to predetermined conditions (eg exposure to UV light), and which are particularly suitable for use in oxygen scavenging packaging.

### DISCLOSURE OF THE INVENTION

Thus, in a first aspect, the present invention provides a method of scavenging oxygen (particularly ground state oxygen) in an atmosphere or liquid comprising the steps of:

(i) treating an anthraquinone compound according to the following formula:

Formula (I)

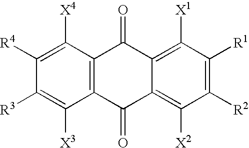

wherein;

X¹, X², X³ and X⁴ are each independently selected from H, C₁-C₂₀ alkoxy, C₁-C₂₀ alkanoyl, C₁-C₂₀ hydroxyalkoxy, C₁-C₂₀ aminoalkoxy, C₁-C₂₀ alkylamido, C₁-C₂₀ alkylcarboxy, C₁-C₂₀ alkylsulfonyl, C₁-C₂₀ alkyl sulfonamido, and sulfonate substituents, and

R¹, R², R³ and R⁴ are each independently selected from H, C₁-C₂₀ alkyl, C₁-C₂₀ alkoxy, C₁-C₂₀ alkanoyl, C₁-C₂₀ alkylamido, C₁-C₂₀ alkylcarboxy, C₁-C₂₀ alkylsulfonyl, C₁-C₂₀ alkyl sulfonamido, sulfonate substituents and L-R⁵ wherein L is selected from O, CH(R⁶) wherein R⁶ is H or C₁-C₆ alkyl, CO₂, CO, SO₃ or SO_{2,} and R₅ is selected from C₁-C₂₀ aminoalkyl, C₁-C₂₀ morpholinoalkyl, C₁-C₂₀ piperazinylalkyl, C₁-C₂₀ alkanol and the radicals represented by,

—CH₂—CH₂(OCH₂CH₂)ₙOH

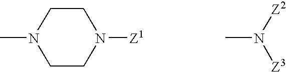

wherein n is any integer between 1 and 20, Z¹ and Z² are selected from H, C₁-C₂₀ alkyl, C₁-C₂₀ alkanol, C₁-C₂₀ aminoalkyl and

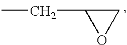

and the radical represented by,

—CH₂—CH₂(OCH₂CH₂)ₙOH

wherein n is as defined above, and Z³ is selected from C₁-C₂₀ alkanol, C₁-C₂₀ aminoalkyl, C₁-C₂₀ morpholinoalkyl, C₁-C₂₀ piperazinylalkyl, and the radical represented by,

—CH₂—CH₂(OCH₂CH₂)ₙOH

wherein n is as defined above, with the proviso that at least one of R¹, R², R³ and R⁴ is/are L-R⁵;

or a salt thereof, or a composition including said anthraquinone compound or salt thereof, with predetermined conditions so as to reduce the anthraquinone compound or salt thereof to a reduced form oxidizable by oxygen; and

(ii) exposing the atmosphere or liquid to said composition; such that at least a portion of the oxygen in the atmosphere or liquid is removed through oxidation of the reduced form of the anthraquinone compound or salt thereof.

Preferably, X¹, X², X³ and X⁴ are each independently selected from H, C₁-C₆ alkoxy, C₁-C₆ alkanoyl, C₁-C₆ hydroxyalkoxy, C₁-C₆ aminoalkoxy, C₁-C₆ alkylamido, C₁-C₆ alkylcarboxy, C₁-C₆ alkylsulfonyl, C₁-C₆ alkyl sulfonamido, and sulfonate substituents.

Preferably, L is selected from O, CH(R⁶), CO and SO₂.

More preferably, L is selected from CO and SO₂.

Preferably, R⁵ is selected from C₁-C₆ aminoalkyl, C₁-C₆ morpholinoalkyl, C₁-C₆ piperazinylalkyl, C₁-C₆ alkanol and the radicals represented by,

—CH₂—CH₂(OCH₂CH₂)ₙOH

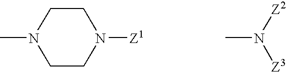

wherein, n is preferably any integer between 1 and 6, Z¹ and Z² are preferably selected from H, C₁-C₆ alkyl, C₁-C₆ alkanol, C₁-C₆ aminoalkyl and

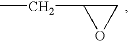

and the radical represented by,

—CH₂—CH₂(CH₂CH₂)ₙOH

wherein n is preferably any integer between 1 and 6, and Z³ is preferably selected from C₁-C₆ alkanol, C₁-C₆ aminoalkyl, C₁-C₆ morpholinoalkyl, C₁-C₆ piperazinylalkyl and the radical represented by,

—CH₂—CH₂(OCH₂CH₂)ₙOH

wherein n preferably any integer between 1 and 6.

More preferably, R⁵ is selected from C₁-C₆ aminoalkyl, C₁-C₆ morpholinoalkyl, C₁-C₆ piperazinylalkyl and the radicals represented by,

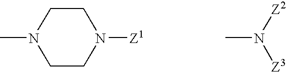

Z¹ and Z² are preferably selected from H, C₁-C₆ alkyl, C₁-C₆ alkanol, C₁-C₆ aminoalkyl and

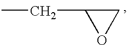

and Z³ is preferably selected from C₁-C₆ aminoalkyl, C₁-C₆ morpholinoalkyl and C₁-C₆ piperazinylalkyl.

Preferably, R¹ is L-R⁵.

Most preferably, the compound is selected from the group consisting of 2,6-bis(3-hydroxypropoxy)anthraquinone, 2,6-bis[2-(2-(2-hydroxyethoxy)ethoxy)ethoxy]-anthraquinone, 2,6-bis(2-morpholino-ethoxy)anthraquinone, 2,6-bis[2-(diethylamino)ethoxy]-anthraquinone, N-(3-morpholinopropyl)-2-anthraquinonesulfonamide, 2-(4methylpiperazine-1-sulfonyl)anthraquinone, N,N′-bis(2-morpholinoethyl)-2,6-anthraquinonedisulfonamide, [N-glycidyl-N-(3-morpholinopropyl)]-2-anthraquinonesulfonamide, 2-(piperazine-1-sulfonyl)-anthraquinonesulfonamide, 2-(1-piperazin-1-yl-ethyl)-anthraquinone, 2-[1-(4methyl-piperazin-1-yl)-ethyl]-anthraquinone and salts thereof.

The steps (i) and (ii) may be carried out in either order.

Step (i) may involve treatment of the anthraquinone compound (or salt thereof) with, for example, light of a certain intensity or wavelength (eg UV light) or, alternatively, the application of heat, γ-irradiation, corona discharge or an electron beam. The reduced anthraquinone compound (or salt thereof) is reactive towards molecular oxygen to produce activated species such as hydrogen peroxide, hydroperoxy radical or a superoxide ion.

Where the anthraquinone compound (or salt thereof) or, alternatively, a composition including said anthraquinone compound (or salt thereof), forms or is incorporated in a packaging material, the exposure of step (ii) may be effected by a step of packing a product (eg a food or beverage) within said packaging material. In the case, where the packaging material is provided in the form of a container, the packing step may generate said atmosphere (eg generation of a “headspace”).

Preferably, the method of the first aspect employs a composition including said anthraquinone compound (or salt thereof). Such a composition also preferably comprises an activated oxygen scavenging agent (ie an agent which reacts with activated oxygen species such as peroxide). Suitable activated oxygen scavenging agents include organic antioxidants, organic phosphites, organic phosphines, organic phosphates, hydroquinone and substituted hydroquinone; inorganic compounds including sulphates, sulphites, phosphites and nitrites of metals; sulphur-containing compounds including thiodipropionic acid and its esters and salts, thio-bis (ethylene glycol beta-aminocrotonate), cysteine, cystine and methionine; and nitrogen-containing compounds including primary, secondary and tertiary amines and their derivatives.

Compositions employed in the method of the first aspect may be in a solid, semi-solid (eg a gel) or liquid form. They may therefore be applied as, or incorporated in, for example, bottle closure liners, inks, coatings, adhesives (eg polyurethanes), films, sheets or layers in containers such as trays or bottles either alone or as laminations or co-extrusions. When used in films or layers, they may be blended with typical polymers and/or copolymers used for construction of films or layers such as those approved for food contact. Such films or layers may be produced by extrusion at temperatures between 50° C. and 350° C. depending upon chemical composition and molecular weight distribution.

In a second aspect, the present invention provides a reaction product of the anthraquinone compound (or salt thereof) defined in the first aspect (such as compounds of the formula VI and IX-XI as shown in FIG. 1), and a compound containing one or more functional groups.

Preferably, the functional compound contains one or more amine, acid, anhydride, alcohol, phenol, thiol, sulfonamide or glycidyl groups. One example is the reaction product of compound of formula VI and poly(ethylene-co-glycidyl methacrylate). A second example is the reaction product of compound of formula IX and poly(ethylene-co-acrylic acid).

The reaction product of the second aspect may provide a means of anchoring the anthraquinone compound (or salt thereof) to a polymer which would be useful in applications where migration of the anthraquinone compound (or salt thereof) may be a concern (eg use of the anthraquinone (or salt thereof) in an oxygen scavenging composition used in direct contact with a food or beverage).

The reaction product of the second aspect may provide a polymer capable of scavenging oxygen when used on its own, or when used in combination with other compounds and/or substances to provide an oxygen scavenging composition.

Thus, in a third aspect, the present invention provides an oxygen scavenging composition comprising a reaction product according to the second aspect.

As with compositions employed in the method of the first aspect, compositions of the third aspect also preferably comprise an activated oxygen scavenging agent, ie an agent which reacts with activated oxygen species such as peroxide. Suitable activated oxygen scavenging agents include organic antioxidants, organic phosphites, organic phosphines, organic phosphates, hydroquinone and substituted hydroquinone; inorganic compounds including sulphates, sulphites, phosphites and nitrites of metals; sulphur-containing compounds including thiodipropionic acid and its esters and salts, thio-bis (ethylene glycol beta-aminocrotonate), cysteine, cystine and methionine; and nitrogen-containing compounds including primary, secondary and tertiary amines and their derivatives.

Also, compositions of the third aspect may be in a solid, semi-solid (eg a gel) or liquid form. They may therefore be applied as, or incorporated in, for example, bottle closure liners, inks, coatings, adhesives (eg polyurethanes), films, sheets or layers in containers such as trays or bottles either alone or as laminations or co-extrusions. When used in films or layers, they may be blended with typical polymers and/or copolymers used for construction of films or layers such as those approved for food contact. Such films or layers may be produced by extrusion at temperatures between 50° C. and 350° C. depending upon chemical composition and molecular weight distribution.

In a fourth aspect, the present invention provides a method for scavenging oxygen (particularly ground state oxygen) in an atmosphere or liquid comprising the steps of:

(i) treating a composition according to the third aspect with predetermined conditions so as to reduce the anthraquinone component(s) of said reaction product to a reduced form oxidizable by oxygen; and

(ii) exposing the atmosphere or liquid to said composition, such that at least a portion of the oxygen in the atmosphere or liquid is removed through oxidation of the reduced form of the anthraquinone component(s).

The steps (i) and (ii) may be carried out in either order.

Numerous specific applications for the anthraquinone compound (or salt thereof) and compositions of the invention are disclosed in Australian Patent No. 672661 as well as in the applicant's co-pending Australian Patent Application No. 87230/98 (the entire disclosure of which is incorporated herein by reference). The anthraquinone compound (or salts thereof) and compositions according to the invention, can also be used to reveal leaks in packages or to indicate package damage caused by handling or tampering. That is, the anthraquinone compound (or salt thereof) and compositions of the invention may undergo an indicative change in colour or change in UV-visible, infrared or near-infrared absorption spectrum, as the capacity for scavenging oxygen becomes exhausted.

Finally, in a fifth aspect, the present invention provides an anthraquinone compound according to the following formula:

Formula (I)

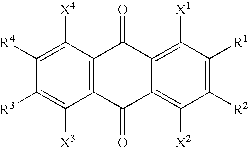

wherein;

X¹, X², X³ and X⁴ are each independently selected from H, C₁-C₂₀ alkoxy, C₁-C₂₀ alkanoyl, C₁-C₂₀ hydroxyalkoxy, C₁-C₂₀ aminoalkoxy, C₁-C₂₀ alkylamido, C₁-C₂₀ alkylcarboxy, C₁-C₂₀ alkylsulfonyl, C₁-C₂₀ alkyl sulfonamido, and sulfonate substituents, and

R¹, R², R³ and R⁴ are each independently selected from H, C₁-C₂₀ alkyl, C₁-C₂₀ alkoxy, C₁-C₂₀ alkanoyl, C₁-C₂₀ akylamido, C₁-C₂₀ alkylcarboxy, C₁-C₂₀ alkylsulfonyl, C₁-C₂₀ alkyl sulfonamido, sulfonate substituents and L-R⁵ wherein L is selected from O, CH(R⁶) wherein R⁶ is H or C₁-C₆ alkyl, CO₂, CO, SO₃ or SO₂ and R⁵ is selected from C₁-C₂₀ aminoalkyl, C₁-C₂₀ morpholinoalkyl, C₁-C₂₀ piperazinylalkyl, C₁-C₂₀ alkanol and the radicals represented by,

—CH₂—CH₂(OCH₂CH₂)ₙOH

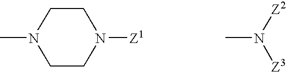

wherein n is any integer between 1 and 20, Z¹ and Z² are selected from H, C₁-C₂₀ alkyl, C₁-C₂₀ alkanol, C₁-C₂₀ aminoalkyl, and

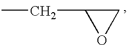

and the radical represented by,

—CH₂—CH₂(OCH₂CH₂)ₙOH

wherein n is any integer between 1 and 20, and Z³ is selected from C₁-C₂₀ alkanol, C₁-C₂₀ aminoalkyl C₁-C₂₀ morpholinoalkyl, C₁-C₂₀ piperazinylalkyl, and the radical represented by,

—CH₂—CH₂(CH₂CH₂)ₙOH

wherein n is any integer between 1 and 20, with the proviso that at least one of R¹, R², R³ and R⁴ is/are L-R⁵; or a salt thereof, wherein said anthraquinone compound or salt thereof is not 2,6-bis(2-morpholino-ethoxy)-anthraquinone, 2,6-bis[2-(diethylamino)ethoxy]-anthraquinone or N,N′-bis(2-morpholinoethyl)-2,6-anthraquinonedisulfonamide.

The terms “comprise”, “comprises” and “comprising” as used throughout the specification are intended to refer to the inclusion of a stated step, component or feature or group of steps, components or features with or without the inclusion of a further step, component or feature or group of steps, components or features.

In the specification, unless stated otherwise, where a document, act or item of knowledge is referred to or discussed, -that reference or discussion is not an admission that the document, act or item of knowledge, or any combination thereof, at the priority date, was part of the common general knowledge in the art.

The invention will now be described with reference to the following, non-limiting examples and accompanying figure(s).

### BRIEF DESCRIPTION OF THE ACCOMPANYING FIGURE(S)

FIG. 1 provides structural formulae II to XII of the compounds described in the following examples.

FIG. 2 provides graphical results showing oxygen scavenging by a composition comprising N-(3-morpholinopropyl)-2-anthraquinonesulfonamide (formula VI) and polyethylene terephthalate (PET), as described in Example 11.

### EXAMPLES

### Example 1

### 2,6-bis(3hydroxypropoxy)-anthraquinone

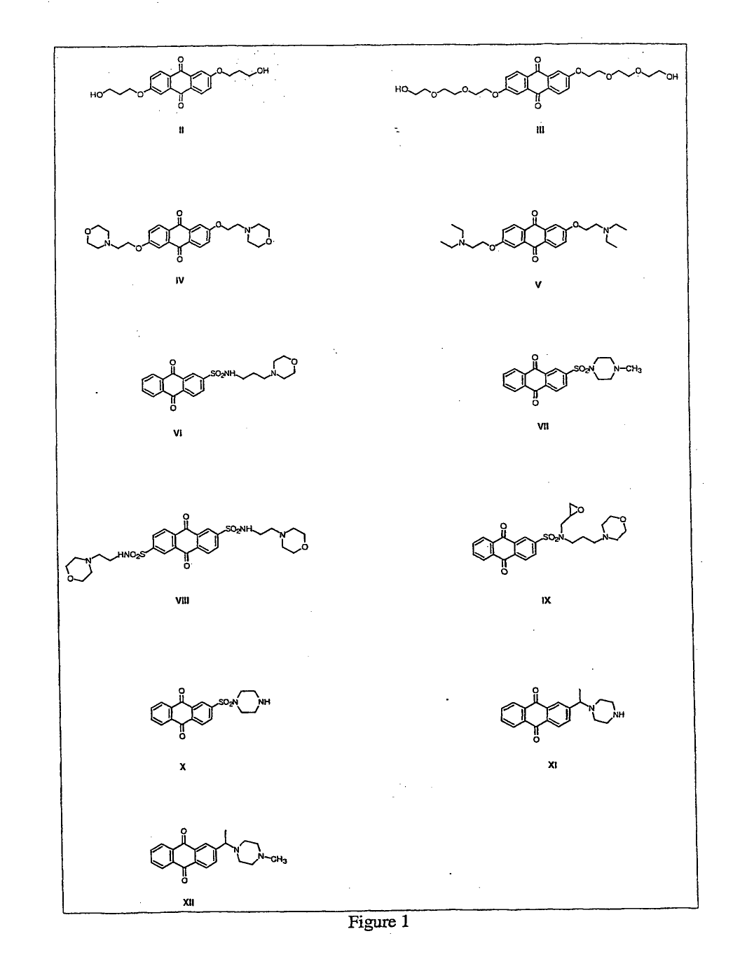

A mixture of 2,6-dihydroxyanthraquinone (2 g, 8.3 mmol), 3-chloropropanol (8 ml, 95.7 mmol), sodium iodide (12.45 g, 83 mmol) and anhydrous K₂CO₃ (11.5 g, 83.2 mmol) in DMF (100 ml) was refluxed for 20 hours under nitrogen. The reaction mixture was then poured into water and the resulting precipitate collected by filtration. The product was washed on the filter with water and methanol, and dried in vacuo (2.46 g, 83%). The product was further purified by recrystallisation from ethanol to give the desired product as a green solid; m.p. 190° C.; ¹H NMR (200 MHz, DMSO-d₆) 1.92 (4H, m, CH₂), 3.59 (4H, m, CH₂OH), 4.23 (4H, m, AQOCH₂), 4.62 (2H, m, OH), 7.35 (2H, d, AOH), 7.51 (2H, s, AOH), 8.07 (2H, d, AOH) ppm. The structure of this compound is shown as formula II in FIG. 1.

### Example 2

### 2,6-bis[2-(2-(2-hydroxyethoxy)ethoxy)ethoxy]-anthraquinone

A mixture of 2,6-dihydroxyanthraquinone (5.69 g, 23.7 mmol), 2-[2-(2-chloroethoxy)ethoxy]ethanol (20 g, 118 mmol), sodium iodide (18 g, 120 mmol) and anhydrous K₂CO₃ (16.35 g, 118 mmol) in DMF (100 ml) was refluxed for 20 hours under nitrogen. The reaction mixture was then poured into water and extracted with chloroform. The organic extracts were combined, washed with water and saturated brine, and dried over MgSO₄. Concentration in vacuo and recrystallisation from ethanol gave the desired product as a yellow solid (2.9 g, 25%); m.p. 110° C. ¹H NMR (200 MHz, CDCl₃) 3.7 (16H, m, OCH₂), 4.0 (4H, t, CH₂OH), 4.40 (4H, t, AQOCH₂), 7.3 (2H, dd, AOH), 7.75 (2H, d, AOH), 8.25 (2H, d, AOH) ppm. The structure of this compound is shown as formula III in in FIG. 1.

### Example 3

### 2,6-bis(2-morpholino-ethoxy)-anthraquinone

4-(2-Chloroethyl)morpholine hydrochloride (3.1 g, 16.7 mmol) was dissolved in water (5 ml), and xylene (6 ml) was then added. The mixture was cooled in an ice bath and whilst being vigorously stirred, a solution of KOH (1.455 g, 26 mmol) in water (1 ml) was added. The xylene layer was decanted and the aqueous layer then extracted with fresh xylene (5 ml). The organic fractions were combined, dried over MgSO₄ and then filtered.

To a solution of 2,6dihydroxyanthraquinone (1 g, 4.2 mmol) in DMF (30 ml) at ˜50° C., was added sodium hydride. (0.21 g, 8.7 mmol). The resulting red coloured reaction mixture was left to stir at 80° C. under nitrogen for 0.5 hour. The free base, 4-(2-chloroethyl)morpholine prepared as above in xylene, was then added, and the reaction mixture was left to stir overnight By TLC analysis (20% MeOH: CHCl₃), all of the starting material had disappeared and two new spots were observed. The warm reaction mixture was poured into water (150 ml), the precipitate collected by filtration and washed with hot water. The grey-brown product was recrystallised from a mixture of methanol (100 ml) and chloroform (50 ml) to give the product as a yellow coloured solid (0.5 g, 26%); m.p. 200-201° C. ¹H NMR (200 MHz, CDCl₃) 2.6 (8H, q, CH₃CH₂N), 2.9 (4H, t, CH₂N), 3.7 (8H, t, CH₂O), 4.3 (4H, t, CH₂OAQ), 7.25 (2H, dd, AOH), 7.7 (2H, d, AOH), 8.25 (2H, d, AOH) ppm. The structure of this compound is shown as formula IV in FIG. 1.

### Example 4

### 2,6bis[2-(diethylamino)ethoxy]-anthraquinone

2-Diethyl aminoethylchloride hydrochloride (2.778 g, 16.1 mmol) was dissolved in water (2.5 ml), and then xylene (6 ml) was added. The mixture was cooled in an ice bath and whilst being vigorously stirred, a solution of KOH (1.432 g, 25.6 mmol) in water (1 ml) was added. The xylene layer was decanted, and the aqueous layer then extracted with fresh xylene (5 ml). The organic fractions were combined, dried over MgSO₄ and then filtered.

To a solution of 2,6-dihydroxyanthraquinone (1 g, 4.2 mmol) in DMF (30 ml) at ˜50° C., was added sodium hydride (0.21 g, 8.7 mmol), and the resulting red coloured reaction mixture stirred at 80° C. under nitrogen for 0.5 hour. The free base, 2-diethylaminoethylchloride in xylene prepared as above, was then added, and the reaction mixture stirred for a further 1 hour. By TLC analysis (20% MeOH:CHCl₃), all of the starting material had disappeared and two new spots were observed. The warm reaction mixture was poured into water (250 ml), the precipitate collected by filtration and washed with hot water. The light brown product was recrystallised from a mixture of methanol (50 ml) and chloroform (15 ml) furnishing a yellow coloured solid (1.042 g, 57%); m.p. 169-170° C. ¹H NMR (200 Mz, CDCl₃) 1.1 (12H, t, CH₃), 2.6 (8H, q, CH₃CH₂N), 2.9 (4H, t, CH₂N), 4.2 (4H, t, CH₂O), 7.2-8.2 (6H, 3×d, AOH) ppm. The structure of this compound is shown as formula V in FIG. 1.

### Example 5

### N-(3morpholinopropyl)-2-anthraquinonesulfonamide

### A. Preparation of 2-anthraquinonesulfonyl chloride

A suspension of 2-anthraquinonesulfonic acid sodium salt (500 g) in thionyl chloride (1 L) was stirred at room temperature for 30 minutes. The suspension was then taken to reflux and DMF (25 ml) added dropwise. After stirring at reflux for 3 hours the excess thionyl chloride was removed in vacuo. The resulting residue was triturated by stirring rapidly in water (5 L), filtered, and the solid isolated and washed with hot water (5 L) followed by methanol (1.5 L), and then air dried prior to drying in vacuo at 40° C. to give 2-anthraquinonesulfonyl chloride (420 g, 90%).

### B. Preparation of N-(3morpholinopropyl)-2-anthraquinonesulfonamide

To a solution of 2-anthraquinonesulfonyl chloride (100 g, 0.33 mol) and 4-(3-aminopropyl)morpholine (95 ml, 0.65 mol) in 2-methoxyethanol (600 ml), was added dropwise over 1 hour a solution of 1M sodium hydroxide in ethanol (341 ml). The reaction mixture was then stirred for 1 hour at room temperature and the resulting precipitate collected, washed successively with methanol, water and further methanol, and finally dried in vacuo at 50° C. to yield (110.8 g, 82%) of N-(3-morpholinopropyl)-2-anthraquinonesulfonamide as a yellow solid; m.p. 151.1-155.7° C. ¹H NMR (200 MHz, CDCl₃) 1.75 (2H, m, CH₂), 2.50 (4H, t, NHCH₂+CH₂N), 3.10 (4H, t, N(CH₂)₂), 3.80 (4H, t, O(CH₂):), 8.20-8.80 (7H, m, AOH) ppm. The structure of tis compound is shown as formula VI in FIG. 1.

### Example 6

### 2-(4methylpiperazine-1-sulfonyl)-anthraquinone

To a solution of 2-anthraquinonesulfonyl chloride (300 g, 0.98 mol) and N-methylpiperazine (217 ml, 1.93 mol) in 2-methoxyethanol (3.6 L), was added dropwise over 2.5 hours a solution of 1M sodium hydroxide in ethanol (986 ml). The reaction mixture was then stirred for 1 hour at room temperature before being diluted with water. The resultant precipitate was collected, washed successively with water and ethanol, and finally dried in vacuo at 50° C. to yield (320 g, 88%) of 2-(4-methylpiperazine-1-sulfonyl)-anthraquinone as a yellow solid; m.p. 215.7-218.3° C. ¹H NMR (200 MHz, d⁶-DMSO) 3.00 (4H, t, N(CH₂)₂), 3.25 (3H, s, CH₃N), 3.65 (4H, t, SO₂N (CH₂)₂), 7.95-8.45 (7H, m, AOH) ppm. The structure of this compound is shown as formula VII in FIG. 1.

### Example 7

### N,N′-bis(2-morpholinoethyl)-2,6-anthraquinonedisulfonamide

### A. Preparation of 2,6-anthraquinonedisulfonyl chloride

A suspension of 2,6-anthraquinonesulfonic acid sodium salt (500 g) in thionyl chloride (1 L) was stirred at room temperature for 30 minutes. The suspension was then taken to reflux and DMF (25 ml) added dropwise. After stirring at reflux for 3 hours the excess thionyl chloride was removed in vacuo. The resulting residue was triturated by stirring rapidly in water (5 L), filtered, and the solid isolated and washed with hot water (5 L) followed by methanol (1.5 L), and then air dried prior to drying in vacuo at 40° C. to give 2-anthraquinonesulfonyl chloride (420 g, 90%).

### B. Preparation of N,N′-bis(2-morpholinoethyl)-2,6-anthraquinonedisulfonamide

To a solution of 2,6-anthraquinonedisulfonyl chloride (0.5 g, 1.23 mmol) and 4-(2-aminoethyl)morpholine (0.7 ml, 4.92 mmol) in 2-methoxyethanol (50 ml), was added dropwise over 0.5 hour a solution of 1M sodium hydroxide in ethanol (2.5 ml). The reaction mixture was then stirred for 1 hour at room temperature and the resulting precipitate collected, washed successively with water and methanol, and finally dried in vacuo at 50° C. to yield (620 mg, 85%) of N,N′-bis(2-morpholinoethyl)-2,6-anthraquinonedisulfonamide; m.p. 229.6-233.4° C. The structure of this compound is shown VIII as formula in FIG. 1.

### Example 8

### 2-(piperazine-1-sulfonyl)-anthraquinonesulfonamide

To a solution of 2-anthraquinonesulfonyl chloride (80 g, 0.26 mol) and piperazine (89.6 g, 1.04 mol) in 2-methoxyethanol (480 ml), was added dropwise over 1 hour a solution of 1M sodium hydroxide in ethanol (265 ml). The reaction mixture was then stirred for 1 hour at room temperature before being diluted with water. The resultant precipitate was collected, washed successively with methanol, warm water and further methanol, and finally dried in vacuo at 50° C. to yield (75.4 g, 81%) of 2-(piperazine-1-sulfonyl)-anthraquinonesulfonamide as a yellow solid. ¹H NMR (200 MHz, d⁶-DMSO) 2.75 (4H, t, N(CH₂)₂), 2.90 (4H, t, SO₂N (CH₂)₂), 7.90-8.50 (7H, m, AOH) ppm. The structure of this compound is shown as formula X in FIG. 1.

### Example 9

### 2-(1-piperazin-1-yl-ethyl)-anthraquinone

### A. Preparation of 2-(1-bromoethyl)-anthraquinone

A mixture of 2-ethylanthraquinone (300 g, 1.27 mol), N-bromosuccimide (229 g, 1.28 mol) and benzoyl peroxide (70%, 2.5 g, 7.2 mmol) in CCl₄(2 L) was stirred at reflux for 1.5 hours. On cooling, the precipitate was collected by filtration and washed on the filter with ethanol, hot water and finally ethanol again. Recrystallisation from methanol/benzene (˜1:1) afforded 2-(1-bromoethyl)-anthraquinone as bright yellow crystals (343 g, 80%).

### B. Preparation of 2-(1-piperazin-4-yl-ethyl)-anthraquinone

To a suspension of 2-(1-bromoethyl)-anthraquinone (500 mg, 1.58 mmol) in refluxing ethanol (8 ml) was added piperazine (681 mg, 7.9 mmol) in one portion. The reaction mixture was maintained at reflux for 2.5 h before being diluted three-fold with water. After acidifying to pH 3 with conc. HCl, the reaction mixture was extracted with chloroform. The aqueous phase was then adjusted to pH 10-11 by the addition of 1M sodium carbonate solution and extracted exhaustively with chloroform. The combined organic extracts were dried over MgSO₄ and concentrated in vacuo to give a pale yellow solid. Yield (410 mg, 81%); m.p. 97.7-98.1° C. ¹H NMR (d⁶-DMSO, 310K) 1.31 (2H, d, CH₃CH), 2.35 (4H, m, N(CH₂)₂), 2.75 (4H, overlapping dd, NH(CH₂)₂), 2.90 (1H, s, NH), 3.60 (1H, q, CH₃CH), 7.75-8.20 (7H, m, AQ aromatic). The structure of this compound is shown as formula XI in FIG. 1.

### Example 10

### Preparation of 2-[1-(4-methylpiperazin-1-yl)-ethyl]-anthraquinone

To a suspension of 2-(1-bromoethyl)-anthraquinone (3 g, 9.47 mmol) in refluxing ethanol (48 ml) was added N-methylpiperazine (5.25 ml, 47.4 mmol) in one portion. The reaction mixture was maintained at reflux for 2.5 h before being diluted three-fold with water. After acidifying to pH 3 with conc. HCl, the reaction mixture was extracted with chloroform. The aqueous phase was then adjusted to pH 10-11 by the addition of 1M sodium carbonate solution and extracted exhaustively with chloroform. The combined organic extracts were dried over MgSO₄ and concentrated in vacuo to give a pale yellow solid. Yield (2.84 g, 89%); m.p. 98.9-102° C. ¹H NMR (CDCl₃) 1.41 (2H, d, CH₃CH), 2.45 (8H, m, N(CH₂)₂), 3.60 (1H, q, CH₃CH), 7.75-8.40 (7H, AQ aromatic). The structure of this compound is shown as formula XI in FIG. 1.

### Example 11

### Oxygen Scavenging by Self-Reducing Anthraquinone Compounds in PET

A composition was prepared by blending N-(3morpholinopropyl)-anthraquinonesulfonamide (formula VI) (prepared according to the method described in Example 5) into a commercially available polyethylene terephthalate at a level of 2% w/w. The composition was then compression molded to form a film having a thickness of about 60 μm. This film was placed between two layers of polypropylene film and vacuum-sealed to form a flat package containing essentially no headspace. The package was placed on a conveyor belt moving at 10 m/min and then exposed to light from a commercial UV-curing lamp (model F-300 fitted with a ‘D’ bulb (Fusion Systems Corp., Maryland, USA)). After exposure to the lamp, the package was opened and the film was then quickly transferred into a foil multilayer bag, and this bag was then vacuum-sealed to form a flat package containing essentially no headspace. This foil-lined pouch allows essentially no ingress of oxygen from the atmosphere into the inside of the pouch. Air was then injected into the foil-lined pouch and the pouch stored at 40° C. The oxygen content inside the pouch was measured by gas chromatography. The volume of oxygen scavenged from the contents of two pouches prepared in the manner described is shown in Table 1.

TABLE 1

| | |
|---|---|
| Storage Time | Oxygen content inside pouch (%) |

| (days) | Pouch 1 | Pouch 2 |
|---|---|---|
| 0 | 20.9 | 20.9 |
| 1 | 18.0 | 18.0 |
| 3 | 17.3 | 17.4 |
| 7 | 16.9 | 16.8 |
| 11 | 16.6 | 16.7 |

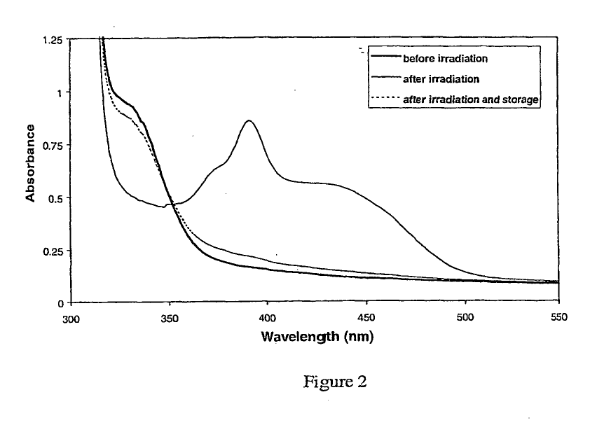

The absorption spectrum of a film prepared from his composition before exposure to the lamp, immediately after exposure to the lamp, and after exposure to the lamp followed by storage in air at 40° C. for 10 days are shown in FIG. 2. These spectra dearly illustrate that photoreduction occurs on exposure to the lamp, followed by re-oxidation upon exposure to air.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

1. A method of scavenging oxygen in an atmosphere or liquid comprising the steps of:
(i) treating an anthraquinone compound according to the following formula:
Formula (I) 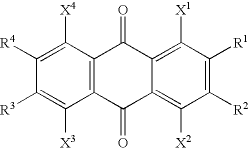
wherein;
X¹, X², X³ and X⁴ are each independently selected from H, C₁-C₂₀ alkoxy, C₁-C₂₀ alkanoyl, C₁-C₂₀ hydroxyalkoxy, C₁-C₂₀ aminoalkoxy, C₁-C₂₀ alkylamido, C₁-C₂₀ alkylcarboxy, C₁-C₂₀ alkylsulfonyl, C₁-C₂₀ alkyl sulfonamido, and sulfonate substituents, and
R¹, R², R³ and R⁴ are each independently selected from H, C₁-C₂₀ alkyl, C₁-C₂₀ alkoxy, C₁-C₂₀ alkanoyl, C₁-C₂₀ alkylamido, C₁-C₂₀ alkylcarboxy, C₁-C₂₀ alkylsulfonyl, C₁-C₂₀ alkyl sulfonamido, sulfonate substituents and L-R⁵ wherein L is selected from O, CH(R⁶) wherein R⁶ is H or C₁-C₆ alkyl, CO₂, CO, SO₃ or SO₂, and R⁵ is selected from C₁-C₂₀ aminoalkyl, C₁-C₂₀ morpholinoalkyl, C₁-C₂₀ piperazinylalkyl, C₁-C₂₀ alkanol and the radicals represented by,
—CH₂—CH₂(OCH₂CH₂)ₙOH 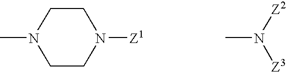
wherein n is any integer between 1 and 20, Z¹ and Z² are selected from H, C₁-C₂₀ alkyl, C₁-C₂₀ alkanol, C₁-C₂₀ aminoalkyl and 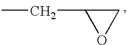
and the radical represented by,
—CH₂—CH₂(OCH₂CH₂)ₙOH
wherein n is any integer between 1 and 20, and Z³ is selected from C₁-C₂₀ alkanol, C₁-C₂₀ aminoalkyl, C₁-C₂₀ morpholinoalkyl, C₁-C₂₀ piperazinylalkyl, and the radical represented by,
—CH₂—CH₂(OCH₂CH₂)ₙOH
wherein n is any integer between 1 and 20, with the proviso that at least one of R¹, R², R³ and R⁴ is/are L-R⁵;
or a salt thereof, or a composition including said anthraquinone one compound or salt thereof, with predetermined conditions so as to reduce the anthraquinone compound or salt thereof to a reduced form oxidizable by oxygen; and
(ii) exposing the atmosphere or liquid to said composition; such that at least a portion of the oxygen in the atmosphere or liquid is removed through oxidation of the reduced form of the anthraquinone compound or salt thereof, and wherein steps (i) and (ii) may be carried out in either order.

2. The method of claim 1, wherein X¹, X², X³ and X⁴ are each independently selected from H, C₁-C₆ alkoxy, C₁-C₆ alkanoyl, C₁-C₆ hydroxyalkoxy, C₁-C₆ aminoalkoxy, C₁-C₆ alkylamido, C₁-C₆ alkylcarboxy, C₁-C₆ alkylsulfonyl, C₁-C₆ alkyl sulfonamido, and sulfonate substituents.

3. The method of claim 1 or 2, wherein L is selected from O, CH(R⁶), CO and SO₂.

4. The method of claim 1 or 2, wherein L is selected from CO and SO₂.

5. The method of claim 1 or 2, wherein R⁵ is selected from C₁-C₆ aminoalkyl, C₁-C₆ morpholinoalkyl, C₁-C₆ piperazinylalkyl, C₁-C₆ alkanol and the radicals represented by,
—CH₂—CH₂(OCH₂CH₂)ₙOH 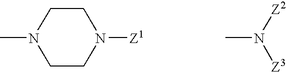
wherein, n is any integer between 1 and 6, Z¹ and Z² are selected from H, C₁-C₆ alkyl, C₁-C₆ alkanol, C₁-C₆ aminoalkyl and 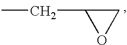
and the radical represented by,
—CH₂—CH₂(OCH₂CH₂)ₙOH
wherein n is any integer between 1 and 6, and Z³ is selected from C₁-C₆ alkanol, C₁-C₆ aminoalkyl, C₁-C₆ morpholinoalkyl and C₁-C₆ piperazinylalkyl, and the radical represented by,
—CH₂—CH₂(OCH₂CH₂)ₙOH
wherein n is any integer between 1 and 6.

6. The method of claim 5, wherein R⁵ is selected from C₁-C₆ aminoalkyl, C₁-C₆ morpholinoalkyl, C₁-C₆piperazinylalkyl and the radicals represented by, 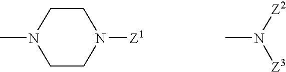
wherein Z¹ and Z² are selected from H, C₁-C₆ alkyl, C₁-C₆ alkanol, C₁-C₆ aminoalkyl and 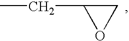
and Z³ is selected from C₁-C₆ aminoalkyl, C₁-C₆ morpholinoalkyl and C₁-C₆ piperazinylalkyl.

7. The method of any one of claims 1 to 6, wherein R¹ is L-R⁵.

8. The method of claim 1, wherein the compound is selected from the group consisting of 2,6-bis(3-hydroxypropoxy)-anthraquinone, 2,6-bis[2-(2-(2-hydroxyethoxy)ethoxy)ethoxy]-anthraquinone, 2,6-bis(2-morpholino-ethoxy)-anthraquinone, 2,6-bis[2-(diethylamino)ethoxy]-anthraquinone, N-(3-morpholinopropyl)-2-anthraquinonesulfonamide, 2-(4methylpiperazine-1-sulfonyl)-anthraquinone, N,N′-bis(3-morpholinoethyl)-2,6-anthraquinonedisulfonamide, [N-glycidyl-N-(3-morpholinopropyl)]-2-anthraquinonesulfonamide, 2-(piperazine-1-sulfonyl)-anthraquinonesulfonamide, 2-(1-piperazin-1-yl-ethyl)-anthraquinone, 2-[1-(4methyl-piperazin-1-yl)-ethyl]-anthraquinone and salts thereof.

9. The method of any one of claims 1 to 8, wherein step (i) involves treatment with UV light.

10. The method of any one of claims 1 to 9, wherein the method employs a composition comprising said anthraquinone compound (or salt thereof), and further comprising an activated oxygen scavenging agent.

11. The method of claim 10, wherein the activated oxygen scavenging agent is selected from the group consisting of organic antioxidants, organic phosphites, organic phosphines, organic phosphates, hydroquinone and substituted hydroquinone, inorganic compounds, sulphur-containing compounds and nitrogen-containing compounds.

12. A reaction product of an anthraquinone compound according to the following formula:
Formula (I) 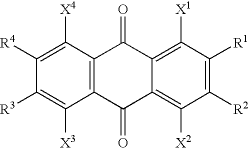
wherein;
X¹, X², X³ and X⁴ are each independently selected from H, C₁-C₂₀ alkoxy, C₁-C₂₀ alkanoyl, C₁-C₂₀ hydroxyalkoxy, C₁-C₂₀ aminoalkoxy, C₁-C₂₀ alkylamido, C₁-C₂₀ alkylcarboxy, C₁-C₂₀ alkylsulfonyl, C₁-C₂₀ alkyl sulfonamido, and sulfonate substituents, and
R¹, R², R³ and R⁴ are each independently selected from H, C₁-C₂₀ alkyl, C₁-C₂₀ alkoxy, C₁-C₂₀ alkanoyl, C₁-C₂₀ alkylamido, C₁-C₂₀ alkylcarboxy, C₁-C₂₀ alkylsulfonyl, C₁-C₂₀ alkyl sulfonamido, sulfonate substituents and L-R⁵ wherein L is selected from O, CH(R⁶) wherein R⁶ is H or C₁-C₆ alkyl, CO₂, CO, SO₃ or SO₂, and R⁵ is selected from C₁-C₂₀ aminoalkyl, C₁-C₂₀ morpholinoalkyl, C₁-C₂₀ piperazinylalkyl, C₁-C₂₀ alkanol and the radicals represented by,
—CH₂—CH₂(OCH₂CH₂)ₙOH 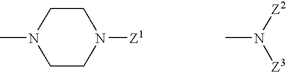
wherein n is any integer between 1 and 20, Z¹ and Z² are selected from H, C₁-C₂₀ alkyl, C₁-C₂₀ alkanol, C₁-C₂₀ aminoalkyl and 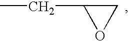
and the radical represented by,
—CH₂—CH₂(OCH₂CH₂)ₙOH
wherein n is any integer between 1 and 20, and Z³ is selected from C₁-C₂₀ alkanol, C₁-C₂₀ aminoalkyl, C₁-C₂₀ morpholinoalkyl and C₁-C₂₀ piperazinylalkyl, and the radical represented by,
—CH₂—CH₂(OCH₂CH₂)ₙOH
wherein n is any integer between 1 and 20, with the proviso that at least one of R¹, R², R³ and R⁴ is/are L-R⁵; or a salt thereof,
and a compound containing one or more functional groups.

13. The reaction product of claim 12, wherein X¹, X², X³ and X⁴ are each independently selected from H, C₁-C₆ alkoxy, C₁-C₆ alkanoyl, C₁-C₆ hydroxyalkoxy, C₁-C₆ aminoalkoxy, C₁-C₆ alkylamido, C₁-C₆ alkylcarboxy, C₁-C₆ alkylsulfonyl, C₁-C₆ alkyl sulfonamido, and sulfonate substituents.

14. The reaction product of claim 12 or 13, wherein L is selected from O, CH(R⁶), CO and SO₂.

15. The reaction product of claim 12 or 13, wherein L is selected from CO and SO₂.

16. The reaction product of claim 12 or 13, wherein R⁵ is selected from C₁-C₆ aminoalkyl, C₁-C₆ morpholinoalkyl, C₁-C₆ piperazinylalkyl, C₁-C₆ alkanol and the radicals represented by,
—CH₂—CH₂(OCH₂CH₂)ₙOH 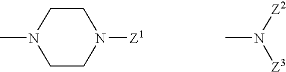
wherein, n is any integer between 1 and 6, Z¹ and Z² are selected from H, C₁-C₆ alkyl, C₁-C₆ alkanol, C₁-C₆ aminoalkyl and 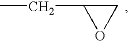
and the radical represented by,
—CH₂—CH₂(OCH₂CH₂)ₙOH
wherein n is any integer between 1 and 6, and Z³ is selected from C₁-C₆ alkanol, C₁-C₆ aminoalkyl, C₁-C₆ morpholinoalkyl, C₁-C₆ piperazinylalkyl and the radical represented by,
—CH₂—CH₂(OCH₂CH₂)ₙOH
wherein n is any integer between 1 and 6.

17. The reaction product of claim 16, wherein R⁵ is selected from C₁-C₆ aminoalkyl, C₁-C₆ morpholinoalkyl, C₁-C₆ piperazinylalkyl and the radicals represented by, 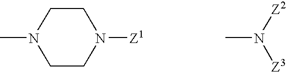
wherein Z¹ and Z² are selected from H, C₁-C₆ alkyl, C₁-C₆ alkanol, C₁-C₆ aminoalkyl and 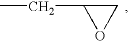
and Z³ is selected from C₁-C₆ aminoalkyl, C₁-C₆ morpholinoalkyl and C₁-C₆piperazinylalkyl.

18. The reaction product of any one of claims 12 to 17, wherein R¹ is L-R⁵.

19. The reaction product of any one of claims 12 to 18, wherein the functional compound contains one or more amine, acid, anhydride, alcohol, phenol, thiol, sulfonamide or glycidyl groups.

20. A reaction product of N-(3morpholinopropyl)-2-anthraquinonesulfonamide and poly(ethylene-co-glycidyl methacrylate).

21. A reaction product of [N-glycidyl-N-(3-morpholinopropyl)]-2-anthraquinonesulfonamide and poly(ethylene-co-acrylic acid).

22. An oxygen scavenging composition comprising a reaction product according to any one of claims 12 to 21.

23. The composition of claim 22, further comprising an activated oxygen scavenging agent.

24. The composition of claim 23, wherein the activated oxygen scavenging agent is selected from the group consisting of organic antioxidants, organic phosphites, organic phosphines, organic phosphates, hydroquinone and substituted hydroquinone, inorganic compounds, sulphur-containing compounds and nitrogen-containing compounds.

25. A method of scavenging oxygen in an atmosphere or liquid comprising the steps of:
(i) treating the composition of any one of claims 22 to 24 with predetermined conditions so as to reduce the anthraquinone component(s) of the reaction product to a reduced form oxidizable by oxygen; and
(ii) exposing the atmosphere or liquid to said composition, such that at least a portion of the oxygen in the atmosphere or liquid is removed through oxidation of the reduced form of the anthraquinone component(s), wherein steps (i) and (ii) may be carried out in either order.

26. The method of claim 25, wherein step (i) involves treatment with UV light.

27. An anthraquinone compound according to the following formula:
Formula (I) 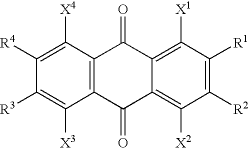
wherein;
X¹, X², X³ and X⁴ are each independently selected from H, C₁-C₂₀ alkoxy, C₁-C₂₀ alkanoyl, C₁-C₂₀ hydroxyalkoxy, C₁-C₂₀ aminoalkoxy, C₁-C₂₀ alkylamido, C₁-C₂₀ alkylcarboxy, C₁-C₂₀ alkylsulfonyl, C₁-C₂₀ alkyl sulfonamido, and sulfonate substituents, and
R¹, R², R³ and R⁴ are each independently selected from H, C₁-C₂₀ alkyl, C₁-C₂₀ alkoxy, C₁-C₂₀ alkanoyl, C₁-C₂₀ alkylamido, C₁-C₂₀ alkylcarboxy, C₁-C₂₀ alkylsulfonyl, C₁-C₂₀ alkyl sulfonamido, sulfonate substituents and L-R⁵ wherein L is selected from O, CH(R⁶) wherein R⁶ is H or C₁-C₆ alkyl, CO₂, CO, SO₃ or SO₂ and R⁵ is selected from C₁-C₂₀ aminoalkyl, C₁-C₂₀ morpholinoalkyl, C₁-C₂₀ piperazinylalkyl, C₁-C₂₀ alkanol and the radicals represented by,
—CH₂—CH₂(OCH₂CH₂)ₙOH 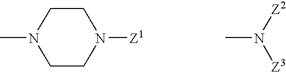
wherein n is any integer between 1 and 20, Z¹ and Z² are selected from H, C₁-C₂₀ alkyl, C₁-C₂₀ alkanol, C₁-C₂₀ aminoalkyl, and 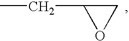
and the radical represented by,
—CH₂—CH₂(OCH₂CH₂)ₙOH
wherein n is any integer between 1 and 20, and Z³ is selected from C₁-C₂₀ alkanol, C₁-C₂₀ aminoalkyl C₁-C₂₀ morpholinoalkyl, C₁-C₂₀ piperazinylalkyl and the radical represented by,
—CH₂—CH₂(OCH₂CH₂)ₙOH
wherein n is any integer between 1 and 20, with the proviso that at least one of R¹, R², R³ and R⁴ is/are L-R⁵; or a salt thereof, wherein said anthraquinone compound or salt thereof is not 2,6-bis(2-morpholino-ethoxy)-anthraquinone, 2,6-bis[2-(diethylamino)ethoxy]-anthraquinone or N,N′-bis(3-morpholinoethyl)-2,6-anthraquinonedisulfonamide.
